(19) United States
(12) Patent Application Publication
(10) Pub. No.: US 2008/0274999 A1
(43) Pub. Date: Nov. 6, 2008

(54) Hyaluronic Acid Fraction with Moisturizing and Anti-Wrinkle Properties

(75) Inventors: Khadija Schwach Abdellaoui, Frederiksberg (DK); Soren Halbye, Vaerlose (DK)

Correspondence Address: NOVOZYMES NORTH AMERICA, INC., 500 FIFTH AVENUE, SUITE 1600, NEW YORK, NY, 10110, US

(73) Assignee: Novozymes Biopolymer A/S, Bagsvaerd (DK)

(21) Appl. No.: 11/722,814

(22) PCT Filed: Dec. 22, 2005
(86) PCT No.: PCT/DK2005/000822

§ 371 Date: Jun. 26, 2007

Related U.S. Application Data
(60) Provisional application No. 60/641,857, filed on Jan. 6, 2005.

(30) Foreign Application Priority Data
Jan. 3, 2005 (DK) PA 2005 00006

(51) Int. Cl. A61K 31/728; A61P 19/02; A61P 17/02; A61P 27/02

(52) U.S. Cl. 514/54

(57) ABSTRACT

A moisturizing, cosmetic, or anti-wrinkle product comprising hyaluronic acid or a salt thereof, wherein the hyaluronic acid or salt thereof has an average molecular weight in the range of 0.7-0.9 MDa, compositions comprising said product, and uses of said product.

### FIELD OF INVENTION

The invention relates to a moisturizing, cosmetic, or anti-wrinkle product comprising hyaluronic acid or a salt thereof, wherein the hyaluronic acid or salt thereof has an average molecular weight in the range of 0.7-0.9 MDa, compositions comprising such a product, and uses of such a product and compositions, e.g. in dermal or transdermal applications and for cosmetic creams or lotions, wherein HA fraction has combined moisturizing and anti-wrinkle properties.

### BACKGROUND

The most abundant heteropolysaccharides of the body are the glycosaminoglycans. Glycosaminoglycans are unbranched carbohydrate polymers, consisting of repeating disaccharide units (only keratan sulphate is branched in the core region of the carbohydrate). The disaccharide units generally comprise, as a first saccharide unit, one of two modified sugars —N-acetylgalactosamine (GalNAc) or N-acetylglucosamine (GlcNAc). The second unit is usually an uronic acid, such as glucuronic acid (GlcUA) or iduronate.

Glycosaminoglycans are negatively charged molecules, and have an extended conformation that imparts high viscosity when in solution. Glycosaminoglycans are located primarily on the surface of cells or in the extracellular matrix. Glycosaminoglycans also have low compressibility in solution and, as a result, are ideal as a physiological lubricating fluid, e.g., joints. The rigidity of glycosaminoglycans provides structural integrity to cells and provides passageways between cells, allowing for cell migration. The glycosaminoglycans of highest physiological importance are hyaluronan, chondroitin sulfate, heparin, heparan sulfate, dermatan sulfate, and keratan sulfate. Most glycosaminoglycans bind covalently to a proteoglycan core protein through specific oligosaccharide structures. Hyaluronan forms large aggregates with certain proteoglycans, but is an exception as free carbohydrate chains form non-covalent complexes with proteoglycans.

Numerous roles of hyaluronan in the body have been identified (see, Laurent T. C. and Fraser J. R. E., 1992, FASEB J. 6: 2397-2404; and Toole B. P., 1991, “Proteoglycans and hyaluronan in morphogenesis and differentiation.” In: Cell Biology of the Extracellular Matrix, pp. 305-341, Hay E. D., ed., Plenum, New York). Hyaluronan is present in hyaline cartilage, synovial joint fluid, and skin tissue, both dermis and epidermis. Hyaluronan is also suspected of having a role in numerous physiological functions, such as adhesion, development, cell motility, cancer, angiogenesis, and wound healing. Due to the unique physical and biological properties of hyaluronan, it is employed in eye and joint surgery and is being evaluated in other medical procedures.

The term “hyaluronic acid” is used in literature to mean acidic polysaccharides with different molecular weights constituted by residues of D-glucuronic and N-acetyl-D-glucosamine acids, which occur naturally in cell surfaces, in the basic extracellular substances of the connective tissue of vertebrates, in the synovial fluid of the joints, in the endobulbar fluid of the eye, in human umbilical cord tissue and in cocks' combs.

The term “hyaluronic acid” is in fact usually used as meaning a whole series of polysaccharides with alternating residues of D-glucuronic and N-acetyl-D-glucosamine acids with varying molecular weights or even the degraded fractions of the same, and it would therefore seem more correct to use the plural term of “hyaluronic acids”. The singular term will, however, be used all the same in this description; in addition, the abbreviation “HA” will frequently be used in place of this collective term.

HA plays an important role in the biological organism, as a mechanical support for the cells of many tissues, such as the skin, tendons, muscles and cartilage, it is a main component of the intercellular matrix. HA also plays other important parts in the biological processes, such as the moistening of tissues, and lubrication.

HA may be extracted from the above mentioned natural tissues, although today it is preferred to prepare it by microbiological methods to minimize the potential risk of transferring infectious agents, and to increase product uniformity, quality and availability (WO 03/0175902, Novozymes).

HA and its various molecular size fractions and the respective salts thereof have been used as medicaments, especially in treatment of arthropathies, as an auxiliary and/or substitute agent for natural organs and tissues, especially in ophtalmology and cosmetic surgery, and as agents in cosmetic preparations. Products of hyaluronan have also been developed for use in orthopaedics, rheumatology, and dermatology.

HA may also be used as an additive for various polymeric materials used for sanitary and surgical articles, such as polyurethanes, polyesters etc. with the effect of rendering these materials biocompatible.

The preparation of a crosslinked HA or salt thereof, which is prepared by crosslinking HA with a polyfunctional epoxy compound is disclosed in EP 0 161 887 B1. Total or partial crosslinked esters of HA with an aliphatic alcohol, and salts of such partial esters with inorganic or organic bases, are disclosed in U.S. Pat. No. 4,957,744.

U.S. Pat. No. 6,673,919 B2 (Chisso Corp. pub. Date Jun. 1, 2004) relates to a process for chemically modifying hyaluronic acid or a salt thereof by O-acetylation, alkoxylation, or crosslinking a complex consisting of hyaluronic acid or a salt thereof and a solution of a cationic compound.

FR 2707653 (Vetoquinol) relates to a conjugate between a biocompatible and biodegradable polymer and a molecule, especially a biologically active molecule containing mobile hydrogen; a process for its preparation; and a pharmaceutical composition including this conjugate.

High molecular weight fractions of HA having an average molecular weight of about 1 to about 1.5 MDa are well known for providing excellent moisturizing properties in cosmetic compositions such as lotions and creams.

Low molecular weight fractions of HA, typically having an average molecular weight of about 0.02 to about 0.4 MDa, have been reported to exhibit anti-wrinkle properties, allegedly due to the ability of these fractions to penetrate the skin barrier.

Both moisturization and anti-wrinkle properties are highly desirable in many applications, and a single compound exhibiting both would be of great commercial interest.

### SUMMARY OF THE INVENTION

The present inventors recently produced HA fractions having an average molecular weight of about 0.7 to about 0.9 MDa, and they evaluated these fractions for both moisturizing and anti-wrinkle effects.

Surprisingly, it was found that HA fractions having an average molecular weight in this range exhibits both moisturizing as well as anti-wrinkle effects.

Accordingly, in a first aspect the invention relates to a moisturizing, cosmetic, or anti-wrinkle product comprising hyaluronic acid or a salt thereof, wherein the hyaluronic acid or salt thereof has an average molecular weight in the range of 0.7-0.9 MDa.

In a second aspect, the invention relates to a composition comprising a product as defined in the first aspect, and an active ingredient, preferably the active ingredient is a pharmacologically active agent.

A third aspect of the invention relates to a pharmaceutical composition comprising an effective amount of a product as defined in the first aspect, together with a pharmaceutically acceptable carrier, excipient or diluent.

A fourth aspect relates to a pharmaceutical composition comprising an effective amount of a product as defined in the first aspect as a vehicle, together with a pharmacologically active agent.

A fifth aspect relates to a cosmetic article comprising as an active ingredient an effective amount of a product as defined in the first aspect.

In a sixth aspect, the invention relates to a sanitary, medical or surgical article comprising a product as defined in the first aspect, preferably the article is a surgical sponge, a wound healing sponge, or a part comprised in a band aid or other wound dressing material.

An important aspect relatest to a medicament capsule or microcapsule comprising a product as defined in the first aspect.

Final aspects of the invention relate to methods of performing procedures in ophtalmology, in the treatment of osteoarthritis or cancer, of treating a wound, of performing dermal or transdermal administration of a pharmacologically active agent, or dermal administration of a cosmetic, the improvement which comprises the use of a product as defined in the first aspect, or a composition as defined in any of the second, third, or fourth aspects.

A number of aspects relate to uses of a product as defined in the first aspect or a composition as defined in any of the preceding aspects, for the manufacture of a medicament for the treatment of osteoarthritis, cancer, the manufacture of a medicament for an ophtalmological treatment, the manufacture of a medicament for the treatment of a wound, the manufacture of a medicament for angiogenesis, or the manufacture of a moisturizer.

### DEFINITIONS

### Nucleic Acid Constructs

“Nucleic acid construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct may be synonymous with the term expression cassette when the nucleic acid construct contains all the control sequences required for expression of a coding sequence. The term “coding sequence” is defined herein as a sequence which is transcribed into mRNA and translated into an enzyme of interest when placed under the control of the below mentioned control sequences. The boundaries of the coding sequence are generally determined by a ribosome binding site located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

The techniques used to isolate or clone a nucleic acid sequence encoding a polypeptide are well known in the art and include, for example, isolation from genomic DNA, preparation from cDNA, or a combination thereof. The cloning of the nucleic acid sequences from such genomic DNA can be effected, e.g., by using antibody screening of expression libraries to detect cloned DNA fragments with shared structural features or the well known polymerase chain reaction (PCR). See, for example, Innis et al., 1990, PCR Protocols: A Guide to Methods and Application, Academic Press, New York. Other nucleic acid amplification procedures such as ligase chain reaction, ligated activated transcription, and nucleic acid sequence-based amplification may be used. The cloning procedures may involve excision and isolation of a desired nucleic acid fragment comprising the nucleic acid sequence encoding the polypeptide, insertion of the fragment into a vector molecule, and incorporation of the recombinant vector into a Bacillus cell where clones of the nucleic acid sequence will be replicated. The nucleic acid sequence may be of genomic, cDNA, RNA, semi-synthetic, synthetic origin, or any combinations thereof.

An isolated nucleic acid sequence encoding an enzyme may be manipulated in a variety of ways to provide for expression of the enzyme. Manipulation of the nucleic acid sequence prior to its insertion into a construct or vector may be desirable or necessary depending on the expression vector or Bacillus host cell. The techniques for modifying nucleic acid sequences utilizing cloning methods are well known in the art. It will be understood that the nucleic acid sequence may also be manipulated in vivo in the host cell using methods well known in the art.

A number of enzymes are involved in the biosynthesis of hyaluronic acid. These enzymes include hyaluronan synthase, UDP-glucose 6-dehydrogenase, UDP-glucose pyrophosphorylase, UDP-N-acetylglucosamine pyrophosphorylase, glucose-6-phosphate isomerase, hexokinase, phosphoglucomutase, amidotransferase, mutase, and acetyl transferase. Hyaluronan synthase is the key enzyme in the production of hyaluronic acid.

“Hyaluronan synthase” is defined herein as a synthase that catalyzes the elongation of a hyaluronan chain by the addition of GlcUA and GlcNAc sugar precursors. The amino acid sequences of streptococcal hyaluronan synthases, vertebrate hyaluronan synthases, and the viral hyaluronan synthase are distinct from the Pasteurella hyaluronan synthase, and have been proposed for classification as Group I and Group II hyaluronan synthases, the Group I hyaluronan synthases including Streptococcal hyaluronan synthases (DeAngelis, 1999). For production of hyaluronan in Bacillus host cells, hyaluronan synthases of a eukaryotic origin, such as mammalian hyaluronan synthases, are less preferred.

The hyaluronan synthase encoding sequence may be any nucleic acid sequence capable of being expressed in a Bacillus host cell. The nucleic acid sequence may be of any origin. Preferred hyaluronan synthase genes include any of either Group I or Group II, such as the Group I hyaluronan synthase genes from Streptococcus equisimilis, Streptococcus pyogenes, Streptococcus uberis, and Streptococcus equi subsp. zooepidemicus, or the Group II hyaluronan synthase genes of Pasturella multocida.

Constructs whereby precursor sugars of hyaluronan are supplied to the host cell are preferably in producing the HA of the invention, either to the culture medium, or by being encoded by endogenous genes, by non-endogenous genes, or by a combination of endogenous and non-endogenous genes in the Bacillus host cell. The precursor sugar may be D-glucuronic acid or N-acetyl-glucosamine.

In the methods of the present invention, the nucleic acid construct may further comprise one or more genes encoding enzymes in the biosynthesis of a precursor sugar of a hyaluronan. Alternatively, the Bacillus host cell may further comprise one or more second nucleic acid constructs comprising one or more genes encoding enzymes in the biosynthesis of the precursor sugar. Hyaluronan production is improved by the use of constructs with a nucleic acid sequence or sequences encoding a gene or genes directing a step in the synthesis pathway of the precursor sugar of hyaluronan. By “directing a step in the synthesis pathway of a precursor sugar of hyaluronan” is meant that the expressed protein of the gene is active in the formation of N-acetyl-glucosamine or D-glucuronic acid, or a sugar that is a precursor of either of N-acetyl-glucosamine and D-glucuronic acid.

In a preferred method for supplying precursor sugars, constructs are provided for improving hyaluronan production in a host cell having a hyaluronan synthase, by culturing a host cell having a recombinant construct with a heterologous promoter region operably linked to a nucleic acid sequence encoding a gene directing a step in the synthesis pathway of a precursor sugar of hyaluronan. In a preferred method the host cell also comprises a recombinant construct having a promoter region operably linked to a hyaluronan synthase, which may use the same or a different promoter region than the nucleic acid sequence to a synthase involved in the biosynthesis of N-acetyl-glucosamine. In a further preferred embodiment, the host cell may have a recombinant construct with a promoter region operably linked to different nucleic acid sequences encoding a second gene involved in the synthesis of a precursor sugar of hyaluronan.

Thus, the present invention also relates to constructs for improving hyaluronan production by the use of constructs with a nucleic acid sequence encoding a gene directing a step in the synthesis pathway of a precursor sugar of hyaluronan. The nucleic acid sequence to the precursor sugar may be expressed from the same or a different promoter as the nucleic acid sequence encoding the hyaluronan synthase.

The genes involved in the biosynthesis of precursor sugars for the production of hyaluronic acid include a UDP-glucose 6-dehydrogenase gene, UDP-glucose pyrophosphorylase gene, UDP-N-acetylglucosamine pyrophosphorylase gene, glucose-6-phosphate isomerase gene, hexokinase gene, phosphoglucomutase gene, amidotransferase gene, mutase gene, and acetyl transferase gene.

In a cell containing a hyaluronan synthase, any one or combination of two or more of hasB, hasC and hasD, or the homologs thereof, such as the Bacillus subtilis tuaD, gtaB, and gcaD, respectively, as well as hasE, may be expressed to increase the pools of precursor sugars available to the hyaluronan synthase. The Bacillus subtilis genome is described in Kunst, et al., Nature 390, 249-256, “The complete genome sequence of the Gram-positive bacterium Bacillus subtilis” (20 Nov. 1997). In some instances, such as where the host cell does not have a native hyaluronan synthase activity, the construct may include the hasA gene.

The nucleic acid sequence encoding the biosynthetic enzymes may be native to the host cell, while in other cases heterologous sequence may be utilized. If two or more genes are expressed they may be genes that are associated with one another in a native operon, such as the genes of the HAS operon of Streptococcus equisimilis, which comprises hasA, hasB, hasC and hasD. In other instances, the use of some combination of the precursor gene sequences may be desired, without each element of the operon included. The use of some genes native to the host cell, and others which are exogenous may also be preferred in other cases. The choice will depend on the available pools of sugars in a given host cell, the ability of the cell to accommodate overproduction without interfering with other functions of the host cell, and whether the cell regulates expression from its native genes differently than exogenous genes.

As one example, depending on the metabolic requirements and growth conditions of the cell, and the available precursor sugar pools, it may be desirable to increase the production of N-acetyl-glucosamine by expression of a nucleic acid sequence encoding UDP-N-acetylglucosamine pyrophosphorylase, such as the hasD gene, the Bacillus gcaD gene, and homologs thereof. Alternatively, the precursor sugar may be D-glucuronic acid. In one such embodiment, the nucleic acid sequence encodes UDP-glucose 6-dehydrogenase. Such nucleic acid sequences include the Bacillus tuaD gene, the hasB gene of Streptococcus, and homologs thereof. The nucleic acid sequence may also encode UDP-glucose pyrophosphorylase, such as in the Bacillus gtaB gene, the hasC gene of Streptococcus, and homologues thereof. In the methods of the present invention, the UDP-glucose 6-dehydrogenase gene may be a hasB gene or tuaD gene; or homologues thereof.

In the present invention it is envisioned that the hyaluronan synthase gene and the one or more genes encoding a precursor sugar are under the control of the same promoter. Alternatively, the one or more genes encoding a precursor sugar are under the control of the same promoter but a different promoter driving the hyaluronan synthase gene. A further alternative is that the hyaluronan synthase gene and each of the genes encoding a precursor sugar are under the control of different promoters. In a preferred embodiment, the hyaluronan synthase gene and the one or more genes encoding a precursor sugar are under the control of the same promoter.

The present invention also relates to a nucleic acid construct comprising an isolated nucleic acid sequence encoding a hyaluronan synthase operon comprising a hyaluronan synthase gene and a UDP-glucose 6-dehydrogenase gene, and optionally one or more genes selected from the group consisting of a UDP-glucose pyrophosphorylase gene, UDP-N-acetylglucosamine pyrophosphorylase gene, and glucose-6-phosphate isomerase gene.

In some cases the host cell will have a recombinant construct with a heterologous promoter region operably linked to a nucleic acid sequence encoding a gene directing a step in the synthesis pathway of a precursor sugar of hyaluronan, which may be in concert with the expression of hyaluronan synthase from a recombinant construct. The hyaluronan synthase may be expressed from the same or a different promoter region than the nucleic acid sequence encoding an enzyme involved in the biosynthesis of the precursor. In another preferred embodiment, the host cell may have a recombinant construct with a promoter region operably linked to a different nucleic acid sequence encoding a second gene involved in the synthesis of a precursor sugar of hyaluronan.

The nucleic acid sequence encoding the enzymes involved in the biosynthesis of the precursor sugar(s) may be expressed from the same or a different promoter as the nucleic acid sequence encoding the hyaluronan synthase. In the former sense, “artificial operons” are constructed, which may mimic the operon of Streptococcus equisimilis in having each hasA, hasB, hasC and hasD, or homologs thereof, or, alternatively, may utilize less than the full complement present in the Streptococcus equisimilis operon. The artificial operons” may also comprise a glucose-6-phosphate isomerase gene (hasE) as well as one or more genes selected from the group consisting of a hexokinase gene, phosphoglucomutase gene, amidotransferase gene, mutase gene, and acetyl transferase gene. In the artificial operon, at least one of the elements is heterologous to one other of the elements, such as the promoter region being heterologous to the encoding sequences.

In a preferred embodiment, the nucleic acid construct comprises hasA, tuaD, and gtaB. In another preferred embodiment, the nucleic acid construct comprises hasA, tuaD, gtaB, and gcaD. In another preferred embodiment, the nucleic acid construct comprises hasA and tuaD. In another preferred embodiment, the nucleic acid construct comprises hasA. In another preferred embodiment, the nucleic acid construct comprises hasA, tuaD, gtaB, gcaD, and hasE. In another preferred embodiment, the nucleic acid construct comprises hasA, hasB, hasC, and hasD. In another preferred embodiment, the nucleic acid construct comprises hasA, hasB, hasC, hasD, and hasE. Based on the above preferred embodiments, the genes noted can be replaced with homologs thereof.

In the methods of the present invention, the nucleic acid constructs comprise a hyaluronan synthase encoding sequence operably linked to a promoter sequence foreign to the hyaluronan synthase encoding sequence. The promoter sequence may be, for example, a single promoter or a tandem promoter.

“Promoter” is defined herein as a nucleic acid sequence involved in the binding of RNA polymerase to initiate transcription of a gene. “Tandem promoter” is defined herein as two or more promoter sequences each of which is operably linked to a coding sequence and mediates the transcription of the coding sequence into mRNA. “Operably linked” is defined herein as a configuration in which a control sequence, e.g., a promoter sequence, is appropriately placed at a position relative to a coding sequence such that the control sequence directs the production of a polypeptide encoded by the coding sequence. As noted earlier, a “coding sequence” is defined herein as a nucleic acid sequence which is transcribed into mRNA and translated into a polypeptide when placed under the control of the appropriate control sequences. The boundaries of the coding sequence are generally determined by a ribosome binding site located just upstream of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acid sequences.

In a preferred embodiment, the promoter sequences may be obtained from a bacterial source. In a more preferred embodiment, the promoter sequences may be obtained from a gram positive bacterium such as a Bacillus strain, e.g., Bacillus agaradherens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

Examples of suitable promoters for directing the transcription of a nucleic acid sequence in the methods of the present invention are the promoters obtained from the E. coli lac operon, Streptomyces coelicolor agarase gene (dagA), Bacillus lentus or Bacillus clausii alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xyIA and xyIB genes, Bacillus thuringiensis subsp. tenebrionis CryliIA gene (cryIIIA) or portions thereof, prokaryotic beta-lactamase gene (VIIIa-Kamaroff et al., 1978, Proceedings of the National Academy of Sciences USA 75:3727-3731). Other examples are the promoter of the spo1 bacterial phage promoter and the tac promoter (DeBoer et al., 1983, Proceedings of the National Academy of Sciences USA 80:21-25). Further promoters are described in “Useful proteins from recombinant bacteria” in Scientific American, 1980, 242:74-94; and in Sambrook, Fritsch, and Maniatus, 1989, Molecular Cloning, A Laboratory Manual, 2d edition, Cold Spring Harbor, N.Y.

The promoter may also be a “consensus” promoter having the sequence TTGACA for the “−35” region and TATAAT for the “−10” region. The consensus promoter may be obtained from any promoter which can function in a Bacillus host cell. The construction of a “consensus” promoter may be accomplished by site-directed mutagenesis to create a promoter which conforms more perfectly to the established consensus sequences for the “10” and “−35” regions of the vegetative “sigma A-type” promoters for Bacillus subtilis (Voskuil et al., 1995, Molecular Microbiology 17: 271-279).

In a preferred embodiment, the “consensus” promoter is obtained from a promoter obtained from the E. coli lac operon, Streptomyces coelicolor agarase gene (dagA), Bacillus clausii or Bacillus lentus alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xyIA and xyIB genes, Bacillus thuringiensis subsp. tenebrionis CryIIIA gene (cryIIIA) or portions thereof, or prokaryotic beta-lactamase gene spo1 bacterial phage promoter. In a more preferred embodiment, the “consensus” promoter is obtained from Bacillus amyloliquefaciens alpha-amylase gene (amyQ).

Widner, et al., U.S. Pat. Nos. 6,255,076 and 5,955,310, describe tandem promoters and constructs and methods for use in expression in Bacillus cells, including the short consensus amyQ promoter (also called scBAN). The use of the cryIIIA stabilizer sequence, and constructs using the sequence, for improved production in Bacillus are also described therein.

Each promoter sequence of the tandem promoter may be any nucleic acid sequence which shows transcriptional activity in the Bacillus cell of choice including a mutant, truncated, and hybrid promoter, and may be obtained from genes encoding extracellular or intracellular polypeptides either homologous or heterologous to the Bacillus cell. Each promoter sequence may be native or foreign to the nucleic acid sequence encoding the polypeptide and native or foreign to the Bacillus cell. The promoter sequences may be the same promoter sequence or different promoter sequences.

The two or more promoter sequences of the tandem promoter may simultaneously promote the transcription of the nucleic acid sequence. Alternatively, one or more of the promoter sequences of the tandem promoter may promote the transcription of the nucleic acid sequence at different stages of growth of the Bacillus cell.

In a preferred embodiment, the tandem promoter contains at least the amyQ promoter of the Bacillus amyloliquefaciens alpha-amylase gene. In another preferred embodiment, the tandem promoter contains at least a “consensus” promoter having the sequence TTGACA for the “−35” region and TATAAT for the “−10” region. In another preferred embodiment, the tandem promoter contains at least the amyL promoter of the Bacillus licheniformis alpha-amylase gene. In another preferred embodiment, the tandem promoter contains at least the cryIIIA promoter or portions thereof (Agaisse and Lereclus, 1994, Molecular Microbiology 13: 97-107).

In a more preferred embodiment, the tandem promoter contains at least the amyL promoter and the cryIIIA promoter. In another more preferred embodiment, the tandem promoter contains at least the amyQ promoter and the cryIIIA promoter. In another more preferred embodiment, the tandem promoter contains at least a “consensus” promoter having the sequence TTGACA for the “−35” region and TATAAT for the “−10” region and the cryIIIA promoter. In another more preferred embodiment, the tandem promoter contains at least two copies of the amyL promoter. In another more preferred embodiment, the tandem promoter contains at least two copies of the amyQ promoter. In another more preferred embodiment, the tandem promoter contains at least two copies of a “consensus” promoter having the sequence TTGACA for the “−35” region and TATAAT for the “−10” region. In another more preferred embodiment, the tandem promoter contains at least two copies of the cryIIIA promoter.

“An mRNA processing/stabilizing sequence” is defined herein as a sequence located downstream of one or more promoter sequences and upstream of a coding sequence to which each of the one or more promoter sequences are operably linked such that all mRNAs synthesized from each promoter sequence may be processed to generate mRNA transcripts with a stabilizer sequence at the 5′ end of the transcripts. The presence of such a stabilizer sequence at the 5′ end of the mRNA transcripts increases their half-life (Agaisse and Lereclus, 1994, supra, Hue et al., 1995, Journal of Bacteriology 177: 3465-3471). The mRNA processing/stabilizing sequence is complementary to the 3′ extremity of a bacterial 16S ribosomal RNA. In a preferred embodiment, the mRNA processing/stabilizing sequence generates essentially single-size transcripts with a stabilizing sequence at the 5′ end of the transcripts. The mRNA processing/stabilizing sequence is preferably one, which is complementary to the 3′ extremity of a bacterial 16S ribosomal RNA. See, U.S. Pat. Nos. 6,255,076 and 5,955,310.

In a more preferred embodiment, the mRNA processing/stabilizing sequence is the Bacillus thuringiensis cryIIIA mRNA processing/stabilizing sequence disclosed in WO 94/25612 and Agaisse and Lereclus, 1994, supra, or portions thereof which retain the mRNA processing/stabilizing function. In another more preferred embodiment, the mRNA processing/stabilizing sequence is the Bacillus subtilis SP82 mRNA processing/stabilizing sequence disclosed in Hue et al., 1995, supra, or portions thereof which retain the mRNA processing/stabilizing function.

When the cryIIIA promoter and its mRNA processing/stabilizing sequence are employed in the methods of the present invention, a DNA fragment containing the sequence disclosed in WO 94/25612 and Agaisse and Lereclus, 1994, supra, or portions thereof which retain the promoter and mRNA processing/stabilizing functions, may be used. Furthermore, DNA fragments containing only the cryIIIA promoter or only the cryIIIA mRNA processing/stabilizing sequence may be prepared using methods well known in the art to construct various tandem promoter and mRNA processing/stabilizing sequence combinations. In this embodiment, the cryIIIA promoter and its mRNA processing/stabilizing sequence are preferably placed downstream of the other promoter sequence(s) constituting the tandem promoter and upstream of the coding sequence of the gene of interest.

The isolated nucleic acid sequence encoding the desired enzyme(s) involved in hyaluronic acid production may then be further manipulated to improve expression of the nucleic acid sequence. Expression will be understood to include any step involved in the production of the polypeptide including, but not limited to, transcription, post-transcriptional modification, translation, post-translational modification, and secretion. The techniques for modifying nucleic acid sequences utilizing cloning methods are well known in the art.

A nucleic acid construct comprising a nucleic acid sequence encoding an enzyme may be operably linked to one or more control sequences capable of directing the expression of the coding sequence in a Bacillus cell under conditions compatible with the control sequences.

The term “control sequences” is defined herein to include all components which are necessary or advantageous for expression of the coding sequence of a nucleic acid sequence. Each control sequence may be native or foreign to the nucleic acid sequence encoding the enzyme. In addition to promoter sequences described above, such control sequences include, but are not limited to, a leader, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding an enzyme.

The control sequence may also be a suitable transcription terminator sequence, a sequence recognized by a Bacillus cell to terminate transcription. The terminator sequence is operably linked to the 3′ terminus of the nucleic acid sequence encoding the enzyme or the last enzyme of an operon. Any terminator which is functional in the Bacillus cell of choice may be used in the present invention.

The control sequence may also be a suitable leader sequence, a nontranslated region of a mRNA which is important for translation by the Bacillus cell. The leader sequence is operably linked to the 5′ terminus of the nucleic acid sequence encoding the enzyme. Any leader sequence which is functional in the Bacillus cell of choice may be used in the present invention.

The control sequence may also be a signal peptide coding region, which codes for an amino acid sequence linked to the amino terminus of a polypeptide which can direct the expressed polypeptide into the cell's secretory pathway. The signal peptide coding region may be native to the polypeptide or may be obtained from foreign sources. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to that portion of the coding sequence which encodes the secreted polypeptide. The foreign signal peptide coding region may be required where the coding sequence does not normally contain a signal peptide coding region. Alternatively, the foreign signal peptide coding region may simply replace the natural signal peptide coding region in order to obtain enhanced secretion of the polypeptide relative to the natural signal peptide coding region normally associated with the coding sequence. The signal peptide coding region may be obtained from an amylase or a protease gene from a Bacillus species. However, any signal peptide coding region capable of directing the expressed polypeptide into the secretory pathway of a Bacillus cell of choice may be used in the present invention.

An effective signal peptide coding region for Bacillus cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis prsA gene. Further signal peptides are described by Simonen and Palva, 1993, Microbiological Reviews 57:109-137.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE) and Bacillus subtilis neutral protease (nprT).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems.

### Production

In the methods of the present invention, the host cells are cultivated in a nutrient medium suitable for production of the hyaluronic acid using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the enzymes involved in hyaluronic acid synthesis to be expressed and the hyaluronic acid to be isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The secreted hyaluronic acid can be recovered directly from the medium.

The resulting hyaluronic acid may be isolated by methods known in the art. For example, the hyaluronic acid may be isolated from the nutrient medium by conventional procedures including, but not limited to, centrifugation, filtration, extraction, spraydrying, evaporation, or precipitation. The isolated hyaluronic acid may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), or extraction (see, e.g., Protein Purification, J. -C. Janson and Lars Ryden, editors, VCH Publishers, New York, 1989).

### FIGURES

FIG. 1: Short term evaluation of hydration. The active cream induce a highly significant variation in the mean basal value of the skin hydration at all times.

FIG. 2: Long term evaluation of hydration. A significant increase of 7% in hydration are recorded after 8 weeks treatment.

FIG. 3: Short term variation of trans epidermal water loss (TEWL) after a single application. The active cream induces a significant variation in the mean basal value of TEWL at 60, 90, 120, 180 min after application.

FIG. 4: Overall elasticity after 8 weeks of application. A significant increase of 27% in the overall elasticity (R2) is observed.

### DETAILED DESCRIPTION OF THE INVENTION

“Hyaluronic acid” is defined herein as an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein.

Rooster combs are a significant commercial source for hyaluronan. Microorganisms are an alternative source. U.S. Pat. No. 4,801,539 discloses a fermentation method for preparing hyaluronic acid involving a strain of Streptococcus zooepidemicus with reported yields of about 3.6 g of hyaluronic acid per liter. European Patent No. EP0694616 discloses fermentation processes using an improved strain of Streptococcus zooepidemicus with reported yields of about 3.5 g of hyaluronic acid per liter. As disclosed in WO 03/054163 (Novozymes), which is incorporated herein in its entirety, hyaluronic acid or salts thereof may be recombinantly produced, e.g., in a Gram-positive Bacillus host.

Hyaluronan synthases have been described from vertebrates, bacterial pathogens, and algal viruses (DeAngelis, P. L., 1999, Cell. Mol. Life. Sci. 56: 670-682). WO 99/23227 discloses a Group I hyaluronate synthase from Streptococcus equisimilis. WO 99/51265 and WO 00/27437 describe a Group II hyaluronate synthase from Pasturella multocida. Ferretti et al. disclose the hyaluronan synthase operon of Streptococcus pyogenes, which is composed of three genes, hasA, hasB, and hasC, that encode hyaluronate synthase, UDP glucose dehydrogenase, and UDP-glucose pyrophosphorylase, respectively (Proc. Natl. Acad. Sci. USA. 98, 4658-4663, 2001). WO 99/51265 describes a nucleic acid segment having a coding region for a Streptococcus equisimilis hyaluronan synthase.

Since the hyaluronan of a recombinant Bacillus cell is expressed directly to the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. If desired, the remaining supernatant may then be filtered, such as by ultrafiltration, to concentrate and remove small molecule contaminants from the hyaluronan. Following removal of the cells and cellular debris, a simple precipitation of the hyaluronan from the medium is performed by known mechanisms. Salt, alcohol, or combinations of salt and alcohol may be used to precipitate the hyaluronan from the filtrate. Once reduced to a precipitate, the hyaluronan can be easily isolated from the solution by physical means. The hyaluronan may be dried or concentrated from the filtrate solution by using evaporative techniques known to the art, such as lyophilization or spraydrying.

The first aspect of the invention relates to a moisturizing, cosmetic, or anti-wrinkle product comprising hyaluronic acid or a salt thereof, wherein the hyaluronic acid or salt thereof has an average molecular weight in the range of 0.7-0.9 MDa.

### Host Cells

A preferred embodiment relates to the product of the first aspect, wherein the hyaluronic acid or salt thereof is recombinantly produced, preferably by a Gram-positive bacterium or host cell, more preferably by a bacterium of the genus Bacillus.

The host cell may be any Bacillus cell suitable for recombinant production of hyaluronic acid. The Bacillus host cell may be a wild-type Bacillus cell or a mutant thereof. Bacillus cells useful in the practice of the present invention include, but are not limited to, Bacillus agaraderhens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Mutant Bacillus subtilis cells particularly adapted for recombinant expression are described in WO 98/22598. Non-encapsulating Bacillus cells are particularly useful in the present invention.

In a preferred embodiment, the Bacillus host cell is a Bacillus amyloliquefaciens, Bacillus clausii, Bacillus lentus, Bacillus licheniformis, Bacillus stearothermophilus or Bacillus subtilis cell. In a more preferred embodiment, the Bacillus cell is a Bacillus amyloliquefaciens cell. In another more preferred embodiment, the Bacillus cell is a Bacillus clausii cell. In another more preferred embodiment, the Bacillus cell is a Bacillus lentus cell.

In another more preferred embodiment, the Bacillus cell is a Bacillus licheniformis cell. In another more preferred embodiment, the Bacillus cell is a Bacillus subtilis cell. In a most preferred embodiment, the Bacillus host cell is Bacillus subtilis A164Δ5 (see U.S. Pat. No. 5,891,701) or Bacillus subtilis 168Δ4.

Transformation of the Bacillus host cell with a nucleic acid construct of the present invention may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278).

### Molecular Weight

The level of hyaluronic acid may be determined according to the modified carbazole method (Bitter and Muir, 1962, Anal Biochem. 4: 330-334). Moreover, the average molecular weight of the hyaluronic acid may be determined using standard methods in the art, such as those described by Ueno et al., 1988, Chem. Pharm. Bull. 36, 4971-4975; Wyatt, 1993, Anal. Chim. Acta 272: 1-40; and Wyatt Technologies, 1999, “Light Scattering University DAWN Course Manual” and “DAWN EOS Manual” Wyatt Technology Corporation, Santa Barbara, Calif.

### Salts and Crosslinked HA

A preferred embodiment relates to a product of the first aspect, which comprises an inorganic salt of hyaluronic acid, preferably sodium hyaluronate, potassium hyaluronate, ammonium hyaluronate, calcium hyaluronate, magnesium hyaluronate, zinc hyaluronate, or cobalt hyaluronate.

It has been found that the reaction of sodium hyaluronate with polylactic acid mono- or di-acyl chloride resulted in a linked or crosslinked HA-PLA or HA-PLA-HA product, which showed an intensified peak at 1736 cm⁻¹ on the IR spectrum, when compared to a standard spectrum of the untreated HA or PLA, corresponding to the presence of newly formed polylactic esters in the linked HA-PLA product.

Accordingly, a preferred embodiment relates to the product of the first aspect, wherein the hyaluronic acid or salt thereof comprises esters of a polymeric alpha hydroxy acid, preferably of polylactic acid or glycolic acid.

It has also been found that treatment of a solution of sodium hyaluronate with boric acid resulted in a crosslinked HA-borate hydrogel, which showed new peaks at 1200 and 945 cm⁻¹ on the FT-IR spectrum, when compared to a standard spectrum of the untreated Na-HA, corresponding to the presence of newly formed borate esters in the crosslinked HA-borate hydrogel.

Accordingly, a preferred embodiment relates to the product of the first aspect, wherein the hyaluronic acid or salt thereof comprises borate esters.

In another preferred embodiment of the product of the first aspect, the hyaluronic acid or salt thereof is fully or partially crosslinked with divinylsulfone (DVS).

### Moisturizing and Anti-Wrinkle Effects

As shown in the examples below, a product of the first aspect has a skin moisturizing effect, which in a preferred embodiment is at least 3% over 8 weeks, preferably at least 5%, most preferably at least 7%, when measured as defined below in the examples.

Further, a product of the first aspect is capable of increasing overall skin elasticity, R2, which in a preferred embodiment is increased with at least 10% over 8 weeks, preferably at least 15%, more preferably at least 20%, when measured as defined below in the examples.

### Other Ingredients

In a preferred embodiment, the product of the invention may also comprise other ingredients, preferably one or more active ingredient, preferably one or more pharmacologically active substance, and also preferably a water-soluble excipient, such as lactose.

In another preferred embodiment, the product of the invention may also comprise one or more enzyme(s), preferably a ligase, transferase, oxidoreductase, hydrolase, lyase, and/or an isomerase; more preferably an amylolytic enzyme, a lipolytic enzyme, a proteolytic enzyme, a cellulytic enzyme, an oxidoreductase or a plant cell-wall degrading enzyme, and more preferably an enzyme with an activity selected from the group consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase.

Non-limiting examples of an active ingredient or pharmacologically active substance which may be used in the present invention include protein and/or peptide drugs, such as, human growth hormone, bovine growth hormone, porcine growth hormone, growth hormone releasing hormone/peptide, granulocyte-colony stimulating factor, granulocyte macrophage-colony stimulating factor, macrophage-colony stimulating factor, erythropoietin, bone morphogenic protein, interferon or derivative thereof, insulin or derivative thereof, atriopeptin-III, monoclonal antibody, tumor necrosis factor, macrophage activating factor, interleukin, tumor degenerating factor, insulin-like growth factor, epidermal growth factor, tissue plasminogen activator, factor IIV, factor IIIV, and urokinase.

A water-soluble excipient my be included for the purpose of stabilizing the active ingredient(s), such excipient may include a protein, e.g., albumin or gelatin; an amino acid, such as glycine, alanine, glutamic acid, arginine, lysine and a salt thereof; carbohydrate such as glucose, lactose, xylose, galactose, fructose, maltose, saccharose, dextran, mannitol, sorbitol, trehalose and chondroitin sulphate; an inorganic salt such as phosphate; a surfactant such as TWEEN® (ICI), poly ethylene glycol, and a mixture thereof. The excipient or stabilizer may be used in an amount ranging from 0.001 to 99% by weight of the product.

Several aspects of the invention relate to various compositions and pharmaceutical comprising, a month other constituents, an effective amount of the product as defined in the first aspect, and an active ingredient, preferably the active ingredient is a pharmacologically active agent; a pharmaceutically acceptable carrier, excipient or diluent, preferably a water-soluble excipient, and most preferably lactose.

In addition, aspects of the invention relate to articles comprising a product as defined in the first aspect or a composition as defined in the aspects and embodiments above, e.g., a cosmetic article, a sanitary article, a medical or surgical article. In a final aspect the invention relates to a medicament capsule or microcapsule comprising a product as defined in the first aspect or a composition as defined in other aspects and embodiments of the invention.

Methods of using the Product or Composition

Various aspects of the invention relate to methods of performing treatment procedures, e.g., in the medical field, using a product of the first aspect, or using compositions of the invention.

One aspect relates to a method of performing procedures in ophtalmology, which comprises the use of a product as defined in the first aspect or a composition of the invention.

Another aspect relates to a method of performing procedures in the treatment of osteoarthritis, which comprises the use of a product as defined in the first aspect or a composition of the invention.

Yet another aspect relates to a method of performing procedures in the treatment of cancer, which comprises the use of a product as defined in the first aspect or a composition of the invention.

An aspect relates to a method of performing transdermal or dermal administration of a pharmacologically active agent, which comprises the use of a product as defined in the first aspect or a composition of the invention.

Another aspect relates to a method of performing dermal administration of a cosmetic, which comprises the use of a product or a composition of the invention.

### EXAMPLES

The efficacy tests of HA in cosmetic formulations were performed using the following formulation for the composition denoted “active cream”:

| Ingredients | % w/w |
|---|---|
| Aqua | 74.20 |
| Sodium hyaluronate (HA) | 0.10 |
| Hydrogenated polydecene | 20.00 |
| Steareth-2 | 3.00 |
| Steareth-21 | 1.00 |
| Ceteraryl alcohol | 1.50 |
| Propylene glycol, diazolidinyl urea, | 0.20 |
| Methylparaben, propylparaben | |

The “placebo cream” was composed of the same ingredients, but without HA.

### Example 1

### Moisturizing Effect (Hydration)

Evaluation of the skin surface hydration of test subjects was performed using corneometry, whereby the electrical capacitance of the Stratum Corneum (SC) is determined, which accurately reflects the relative SC moisture. The measurements were performed using a Corneometer: Combi CM 825 (Courage & Khazaka).

The hydration study was performed by applying the creams on the forearms of 12 volunteers (female, mean age 38 years), and then comparing the resulting hydration from application of the active cream and the placebo cream with that of an untreated area.

The hydration was evaluated over a short term (up to 180 min) after the application of the creams, and over a longer term by comparing the results over 8 weeks.

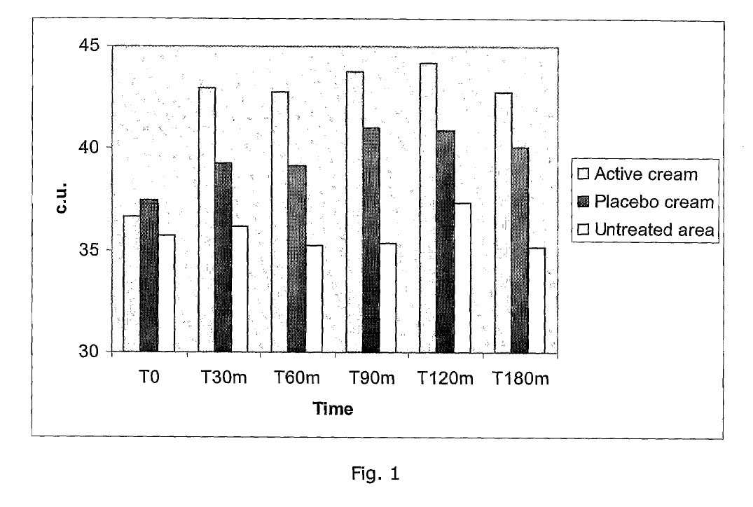

The short term hydration measurements are shown in FIG. 1. The active cream clearly induced a highly significant variation in the mean basal value of the skin hydration at all times.

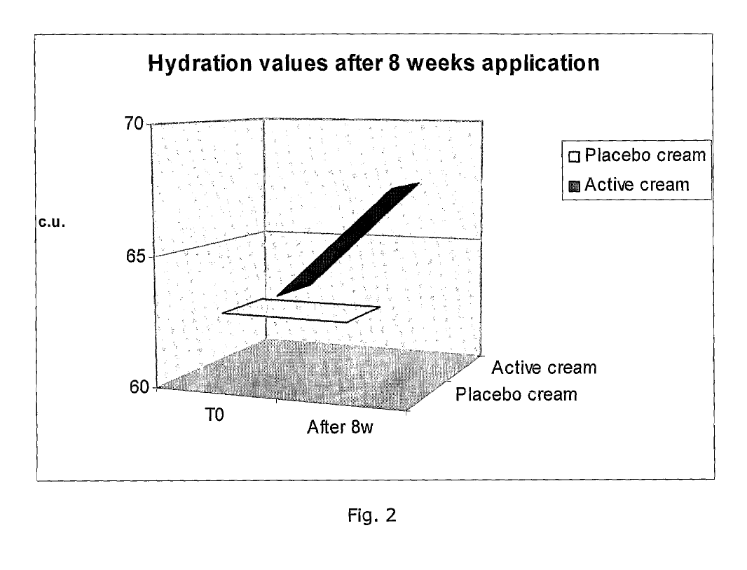

The long term evaluation results are shown in FIG. 2. A significant increase of 7% in hydration was recorded over the 8 weeks of treatment with active cream.

### Example 2

### Trans Epidermal Water Loss (TEWL)

The trans epidermal water loss (TEWL) is an important parameter for evaluating the efficiency of the skin barrier in terms of hydration or moisturization. TEWL measures the water vapor released by the skin surface based on Fick's diffusion formula. The equipment used to measure this is the Tewameter™ 210 (Courage & Khazaka). Measurements were taken on 12 volunteer test subjects after application of the active cream (0.1% HA) and the placebo cream, and also of an untreated skin area.

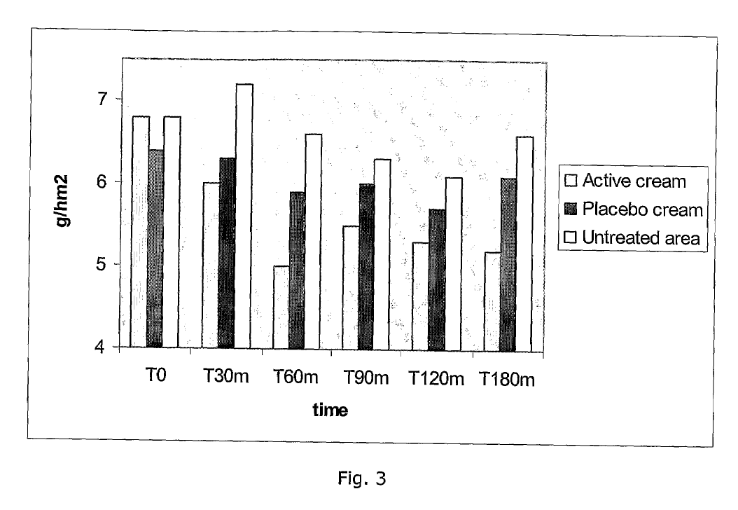

The short term variation of TEWL after a single application is shown in FIG. 3. The active cream induces a significant lowering of the mean basal value of TEWL at 60, 90, 120, and 180 min. after application.

### Example 3

### Anti-Wrinkle Effect (Elasticity)

The anti-wrinkle study was carried out using 12 volunteer human test subjects (female, mean age of 46 years) with application of the creams on the face twice daily over 8 weeks. All tests were performed in a bioclimatic room at 24° C. and 50% relative humidity (RH).

Skin elasticity was measured on the test subjects using a Cutometer SEM 575 (Courage & Khazaka). The Cutometer measures the vertical deformation of the skin, when sucked into the opening of a measuring probe. This method provides the following deformation parameters relating to the skin elasticity: R0 (maximum deformation), R2 (overall elasticity) and R6 (viscoelastic ratio). The elasticity test was performed after each facial cream application during the 8 weeks of treatment with the active cream (comprising 0.1% HA), and the placebo cream.

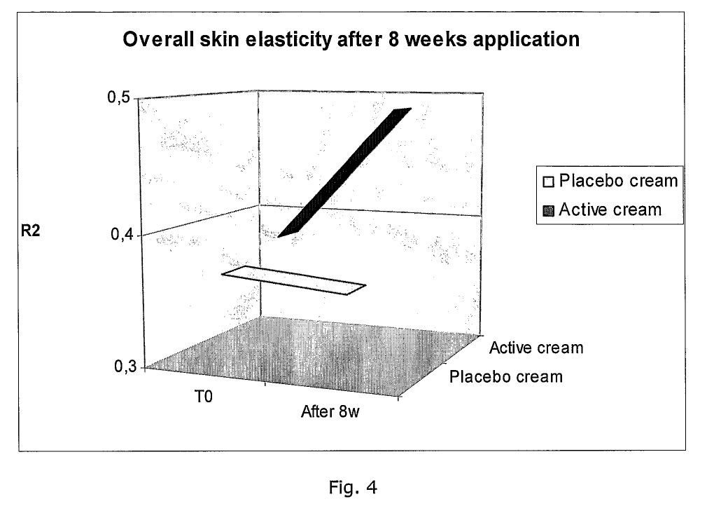

The results are shown in FIG. 4. After 8 weeks of application a significant increase of 27% in the overall elasticity (R2) was observed.

### Example 4

### Skin Topography

The topography of the skin surface is evaluated by skin surface replicas and image analysis. The principle of the test is to obtain a negative imprint of the skin surface by applying a fast hardening synthetic polymer (SILFLO—Flexico Ltd. UK.). This replica is then analyzed by image digitalization. From this image the standard roughness parameters Ra (mean roughness) and Rz (maximum roughness for deep wrinkles) is calculated.

This test was performed after face treatment twice daily over 8 weeks with the active cream in comparison to the placebo cream. In the area treated with the active product the mean maximum roughness value (Rz) significantly decreased by 10% after 8 weeks of treatment. The placebo treated area did not show any variation in the Rz values.

1-31. (canceled)

32. A moisturizing, cosmetic, or anti-wrinkle product comprising hyaluronic acid or a salt thereof, wherein the hyaluronic acid or salt thereof has an average molecular weight in the range of 0.7-0.9 MDa.

33. The product of claim 32, wherein the hyaluronic acid or salt thereof is recombinantly produced.

34. The product of claim 32, wherein the hyaluronic acid or salt thereof is an inorganic salt of hyaluronic acid.

35. The product of claim 32, which has a skin moisturizing effect of at least 3% over 8 weeks.

36. The product of claim 32, which is capable of increasing overall skin elasticity, R2, with at least 10% over 8 weeks.

37. The product of claim 32, wherein the hyaluronic acid or salt thereof comprises esters of boric acid and/or a polymeric alpha hydroxy acid.

38. The product of claim 32, wherein the hyaluronic acid or salt thereof is fully or partially cross-linked with divinylsulfone (DVS).

39. The product of claim 32, which also comprises an active ingredient.

40. The product of claim 32, which also comprises a water-soluble excipient.

41. A composition comprising a product as defined in claim 32 and an active ingredient.

42. The composition of claim 41, which also comprises a water-soluble excipient.

43. A pharmaceutical composition comprising an effective amount of a product as defined in claim 32, together with a pharmaceutically acceptable carrier, excipient or diluent.

44. A pharmaceutical composition comprising an effective amount of a product as defined in claim 32 as a vehicle, together with a pharmacologically active agent.

45. A cosmetic article comprising as an active ingredient an effective amount of a product as defined in claim 32.

46. A sanitary, medical or surgical article comprising a product as defined in claim 32.

47. A medicament capsule or microcapsule comprising a product as defined in claim 32.

48. An ophtamological method of using a product as defined in claim 32, or a composition as defined in claim 41.

49. A method of treating osteoarthritis, comprising administering an effective amount of a product as defined in claim 32 or a composition as defined in claim 41 to a mammal.

50. A method of treating a wound, comprising administering an effective amount of a product as defined in claim 32 or a composition as defined in claim 41 to a mammal.
